(19) United States
(12) Patent Application Publication
(10) Pub. No.: US 2014/0128674 A1
(43) Pub. Date: May 8, 2014

(54) CONTACT-FREE MAGNETIC COUPLING FOR AN ENDOSCOPE, AND ENDOSCOPE

(71) Applicant: OLYMPUS WINTER & IBE GMBH, Hamburg (DE)

(72) Inventor: Martin WIETERS, Hamburg (DE)

(73) Assignee: OLYMPUS WINTER & IBE GMBH, Hamburg (DE)

(21) Appl. No.: 14/150,941

(22) Filed: Jan. 9, 2014

Related U.S. Application Data
(63) Continuation of application No. PCT/EP2012/002768, filed on Jul. 2, 2012.

(30) Foreign Application Priority Data
Jul. 11, 2011 (DE) 102011078969.3

(51) Int. Cl. A61B 1/00; H01F 7/02; A61B 1/04

(52) U.S. Cl. CPC A61B 1/00112; A61B 1/04; H01F 7/0252
USPC 600/109; 600/160; 335/285

(57) ABSTRACT

A contact-free magnetic coupling for an endoscope. The contact-free magnetic coupling including: an outer coupling part; and an inner coupling part. Wherein the inner coupling part is disposed within the outer coupling part such that a gap remains between the outer and inner coupling parts; the outer coupling part and the inner coupling part each comprise an annular body; the annular body of the outer coupling part is disposed between first side anchor plates, which together form a substantially “U”-shaped cross section that is open toward an interior; and/or the annular body of the inner coupling part is disposed between second side anchor plates, which together form a substantially “U”-shaped cross section that is open toward an exterior. Wherein the annular body of the outer coupling part and/or the inner coupling part comprises an axially magnetized annular magnet.

### CROSS-REFERENCE TO RELATED APPLICATION

The present application is a continuation of PCT/EP2012/002768 filed on Jul. 2, 2012, which is based upon and claims the benefit to DE 10 2011 078 969.3 filed on Jul. 11, 2011, the entire contents of each of which are incorporated herein by reference.

### BACKGROUND

1. Field

The invention relates to a contact-free magnetic coupling for an endoscope, in particular a video endoscope, having an outer coupling part and an inner coupling part, wherein the inner coupling part is disposed in the magnetic coupling within the outer coupling part, wherein a gap remains between the coupling parts in the magnetic coupling. The invention further relates to an endoscope, in particular a video endoscope.

2. Prior Art

Contact-free magnetic couplings for endoscopes are known from the prior art. These couplings serve to transfer a movement, using a rotary ring on the handle of an endoscope, to an inner tube of the endoscope shaft without contact thereto. The movement serves to move optical units in the distal region of the endoscope shaft in order to change a viewing direction, for example. The change of viewing direction can be a change of an azimuth angle, that is, a rotation about the longitudinal axis of the endoscope shaft, or a discrete or continuous change of the viewing direction with respect to the polar angle thereof, thus a change of the deviation of the viewing direction from 0° or the straight ahead viewing direction. The change of the azimuth angle occurs with side-viewing endoscopes.

The change of the azimuth angle of the viewing direction goes along with a rotation of an inner tube of the endoscope shaft, at which an optical assembly is secured, which views straight ahead, with respect to an outer tube which is connected to an optical assembly, which causes a deflection of the light from a side viewing direction into a longitudinal axial optical path, for example by means of mirrors and/or prisms.

From the prior art, the “EndoEye” system from the applicant is known with which torque is transferred by means of several bar magnets in a rotary ring on the handle. The same number of bar magnets is disposed in both the handle as well as in the rotary ring, for example, four each. A low number reduces the power of the coupling and a large number increases the power, however, also increases the complexity of the design and the assembly. Here, it is relevant that a specific polarity of the magnets with respect to each other is maintained so that in each case a magnetic circuit is closed by two magnets. An outer and an inner holder are necessary for positioning and housing the magnets. Here, a plurality of components is required for the magnetic coupling; for the “HD EndoEye” concept from the applicant, for example, eight magnets and at least two parts for the mounting are required. For this reason, the costs for the individual parts and the costs for the assembly are relatively high, wherein the necessary alignment of the polarity of the magnets makes the assembly prone to errors. Furthermore, due to the assembly of the magnets on the holders, relatively large deviations can arise in the form and position, which can influence the function, for example, can cause a grinding or jamming.

With the construction used in the “EndoEye” system of the applicant, only torque can be transferred. This system is not suitable for a use for transferring axial forces.

Other endoscopes, in particular video endoscopes having optical assemblies at the distal tip of the longitudinally extended endoscope, have optical elements that in some cases can be moved longitudinally or transversely. This can be used for a longitudinal movement of an optical element, for example for focusing. Transverse movements can serve for example for guiding optical filters in and out of the optical path of the endoscope. For this purpose, actuators are used that create the longitudinal or transverse movement of the optical assemblies.

A further application of axially movable optical elements with endoscopes having a plurality of discrete viewing directions, is to switch back and forth between the different viewing directions. Such endoscopes have at least one sideways viewing direction and a further viewing direction which is also directed to the side or straight ahead. With appropriate endoscopes, by simply switching the viewing direction, the visual field can be greatly expanded in the operative field without the endoscope itself needing to be tilted. Typical combinations of viewing directions in endoscopes having several discrete viewing directions are, for example, 0° and 45° or 30° and 80°. This property is also known as a “changeable direction of view” (“c-DOV”).

With another type of endoscope, a sideways direction of view is set by pivoting or moving a mirror or a prism, or a suitable other optical element or optical elements. The switching occurs here in a quasi-continuous manner because there is a panning of the viewing field instead of a completely discrete switching. A further type of endoscope has a pivotable objective, the viewing direction of which is set directly. This is also referred to as “variable direction of view” (v-DOV”).

From the patent application, DE 10 2011 005 255.0, of the applicant, a distal optical assembly of a video endoscope is known which comprises an actuator and an optical element that can be moved transverse to the longitudinal axis of the video endoscope, wherein the assembly further comprises a diversion device which diverts a longitudinal displacement of the actuator into a direction transverse to the longitudinal axis of the video endoscope and transfers this to the movable optical element. The movable optical element is a mirror and/or a prism, by means of which it is possible to switch back and forth between different sideways viewing directions with respect to the polar angle.

In general, an optical assembly at the distal tip of the endoscope is also called an “R-unit”. It contains the optical lens system and possibly an optical area sensor, a CCD chip or a CMOS chip, for example. Alternatively, the optical assembly can also lead to an optical rod lens system or a system with fiber optics which further conducts the light from the optical assembly to proximally. The light sensitive sensor can then be disposed in a handle or in a camera head, which is arranged at a proximal ocular. These systems are comprised in the scope of the invention.

### SUMMARY

It is the object of the present invention to provide a flexible and structurally easily implementable handling for positioning of assemblies disposed in the interior of the endoscope, which can also be easily and intuitively operated.

This object is achieved by a contact-free magnetic coupling for an endoscope, in particular a video endoscope, having an outer coupling part and an inner coupling part, wherein the inner coupling part is arranged in the magnetic coupling within the outer coupling part, wherein a gap remains between the coupling parts in the magnetic coupling, developed further in that the outer coupling part and the inner coupling part each comprise an annular body, wherein the annular body of the outer coupling part is arranged between side anchor plates, which together form a substantially “U”-shaped cross section that is open toward the interior and/or the annular body of the inner coupling part is arranged between side anchor plates, which together form a substantially “U”-shaped cross section that is open toward the exterior, wherein the annular body of the outer coupling part and/or the inner coupling part comprises an axially magnetized annular magnet.

The invention is based on the fundamental idea that the complexity of the design is reduced in that instead of the bar magnets from the “EndoEye” system of the applicant, an axially magnetized annular magnet is used in order to generate a magnetic field. For bundling the magnetic field, an anchor plate is placed as a pole shoe on each side of the annular magnet. For closing the magnetic circuit, an inner coupling part is used which connects the two pole shoes together and itself also has anchor plates as pole shoes. The roles of the inner coupling part and the outer coupling part can also be exchanged. The annular body which is provided as a connecting part can be implemented as a simple sleeve. This coupling part, however, can in turn also have other lateral disks in order to more strongly bundle the magnetic field lines, and thereby to more efficiently implement the force transfer.

In general, either only the outer coupling part can comprise an annular magnet, or only the inner part can comprise an annular magnet, or both coupling parts each comprise an annular magnet.

In addition, the two coupling parts preferably have substantially the same dimension in the axial direction.

Due to this design according to the invention, it is possible to transfer axial forces from annular magnets having anchor plates to the opposite coupling part. This is because the magnetic flux lines are localized very strongly at discrete axial positions, namely between the tips of the anchor plates and the opposite coupling part. A movement of the outer coupling part in the axial direction therefore leads to an equal movement of the inner coupling part in order to again take on an energetically favorable arrangement of the magnetic field.

In the case that only one annular magnet is used, the coupling part that does not comprise an annular magnet is preferably composed at least partially from a ferromagnetic material, and in particular is preferably one-piece. This also applies to the case that the coupling part which does not comprise an annular magnet does, however, have anchor plates and thus a substantially “U”-shaped cross section.

Alternatively, if the two coupling parts have annular magnets it is preferably provided that the annular magnets are oppositely poled axially to each other.

Further preferably the anchor plates are composed at least to some extent from ferromagnetic material. A ferromagnetic material bundles the magnetic field lines, or respectively flux lines, in the interior thereof and guides them bundled to the exit areas, in particular the tips, or respectively the perimeter, of the anchor plates so that with little expenditure, the shape of the desired magnetic field is set by the selection of the shape of the ferromagnetic components of the coupling parts, and thus an efficient and reliable magnetic force transfer is attained.

In order to also transfer torque, the anchor plates of the two coupling parts, at their respective surfaces bordering the gap between the coupling parts, preferably have a structure in the peripheral direction corresponding to each other with pole shoe segments. The simplest structure consists in each case in a pole shoe segment at the exterior periphery of the anchor plate of the inner coupling part and at the inner periphery of the anchor plate of the outer coupling part. Two or more pole shoe segments can also be provided. The pole shoe segments extend over the respective periphery of the anchor plates and thus lead to a localized concentration of the magnetic flux lines, or respectively flow lines, in a peripheral direction. Thus, the energetically most favorable position of the outer and inner coupling parts to each other is that in which the magnetic field lines between the pole shoe segments of the anchor plates must cover the shortest path through the gap, thus, that is, an arrangement in which the pole shoe segments of the anchor plates of the inner coupling part and the anchor plates of the outer coupling part lie directly over one another. A rotation of the outer coupling part leads therefore directly to a rotation of the inner coupling part.

The contact-free magnetic coupling has the further advantage that no mechanical connection exists between the inner coupling part and the outer coupling part. If the inner tube of the endoscope shaft, which is connected to the inner coupling part, experiences resistance or a limit with respect to the rotation, the outer coupling part can be rotated without the inner coupling part reproducing the rotation beyond the resistance. This represents a built-in safety measure and a built-in protection for the sensitive parts of the endoscope. Therefore, the force of the magnetic coupling is selected so that no forces can be exerted on the inner tube and the optical components connected thereto, of a magnitude which could lead to damage thereof.

In an advantageous embodiment, the two anchor plates of each coupling part have the same shape, and/or are disposed in the same angular relationship to each other. This means for example that each of the two anchor plates of the inner coupling part, or respectively the outer coupling part, at any moment and with any applied force, transfers the force synchronously to the inner coupling part.

In an alternate design, which is also advantageous, the two anchor plates of each coupling part are formed differently, particularly having different numbers of pole shoe segments and/or disposed in different angular relationship to each other. For example, the anchor plates, which are disposed distally to the coupling parts, may have six pole shoe segments, whereas the proximal anchor plates have five or seven pole shoe segments. Alternatively each of the anchor plates can also have six pole shoes, for example, but be rotated by 30° with respect to each other. The pole shoe segments can also have different shapes. This also leads to an equalization of transfer of force. However, it has to be ensured that the anchor plates of the two coupling parts corresponding to each other, are of the same type and are disposed at the same angular relationship to each other. For transferring torque, the two coupling parts must in each case have anchor plates correlated in the peripheral direction in order to avoid otherwise possible unstable position relationships of the coupling parts to each other.

The object of the invention is also achieved by an endoscope, in particular a video endoscope, having a contact-free magnetic coupling according to the invention, described above, which in particular has a switchable or changeable viewing direction and/or a changeable lateral viewing direction.

Such an endoscope is designed to transfer axial forces by means of a mechanically simple and easy handling system in the form of the contact-free magnetic coupling according to the invention that can be used in particular for endoscopes having a variable viewing direction (“v-DOV”) and having discretely changeable viewing direction (“c-DOV”).

The features, properties, and advantages named for the individual invention objects, i.e. the contact-free magnet coupling and the endoscope, also apply without restriction to the respective other invention objects, which relate to each other.

Further characteristics of the invention will become apparent from the description of the embodiments according to the invention together with the claims and the included drawings. Embodiments according to the invention can fulfill individual characteristics or a combination of several characteristics.

### BRIEF DESCRIPTION OF THE DRAWINGS

The invention is described below, without restricting the general intent of the invention, based on exemplary embodiments in reference to the drawings, whereby we expressly refer to the drawings with regard to the disclosure of all details according to the invention that are not explained in greater detail in the text. The figures show

FIG. 1 illustrates a schematic cross sectional representation through an endoscope according to the prior art,

FIG. 2 illustrates a schematic cross sectional representation through a magnet configuration of a magnetic coupling according to the prior art,

FIG. 3 illustrates a schematic perspective representation in section through a contact-free magnetic coupling according to the invention,

FIG. 4 illustrates a schematic cross sectional representation through the magnetic coupling according to the invention according to FIG. 3, and

FIG. 5 illustrates a schematic lateral view of a magnetic coupling according to the invention according to FIG. 3.

### DETAILED DESCRIPTION

In the drawings, the same or similar types of elements and/or parts are denominated with the same reference numbers so that a corresponding re-introduction can be omitted.

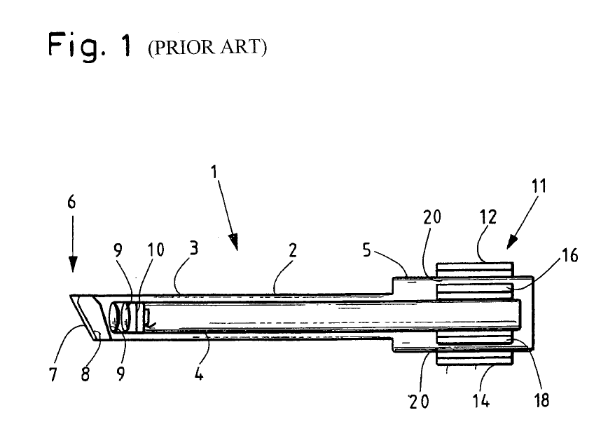

FIG. 1 schematically shows a cross section of an endoscope 1 according to the prior art. The endoscope 1 has a longitudinally extending shaft 2 having an outer tube 3 and an inner tube 4. For the sake of clarity, other possible tubes are not represented. At the proximal end there is a handle 5, at the distal end 6 there is a diagonally positioned, thus sideways looking entry window 7. Attached to the entry window 7, there is a prism unit 8 that diverts the light entering from the side into a longitudinal direction. Entry window 7 and prism unit 8 form an optical assembly that is connected to the outer tube 3. A rotation of the handle 5 and the outer tube 3 with the handle 5 leads therefore to a change of the viewing direction of the endoscope 1 about the longitudinal axis of the endoscope 1, thus about the azimuth angle.

Optical units of an optical assembly, namely lenses 9, 9′, which divert the entering light onto a CCD sensor 10, which receives the incident light and further conducts the light and image data on an electronic path, not represented, to an image representation unit, also not represented, are connected to the inner tube 4 at the distal end 6 of the shaft 2.

The inner tube 4 with the optical assembly having the lenses 9, 9′ and the CCD sensor 10 can be rotated with respect to the outer tube 3 about the longitudinal axis of the endoscope shaft 2. In this way the operator maintains the orientation of the image despite a change of the viewing direction about the azimuth angle.

The handle 5 has a contact-free magnetic coupling 11 which, as with the “EndoEye” system of the applicant, is based on bar magnets 12 to 19. In the section in FIG. 1, two outer bar magnets 12, 14 are shown which can be rotated in a rotational ring about the handle 5, and two bar magnets 16, 18 which are connected to the inner tube 4. The bar magnets 12, 14, 16, 18 are aligned with each other in the axial direction. A rotation of the outer ring with the bar magnets 12, 14 leads to the bar magnets 16, 18 also being rotated with the inner tube 4 such that the inner tube 4 is rotated in the outer tube 3 about the longitudinal axis of the endoscope shaft 2.

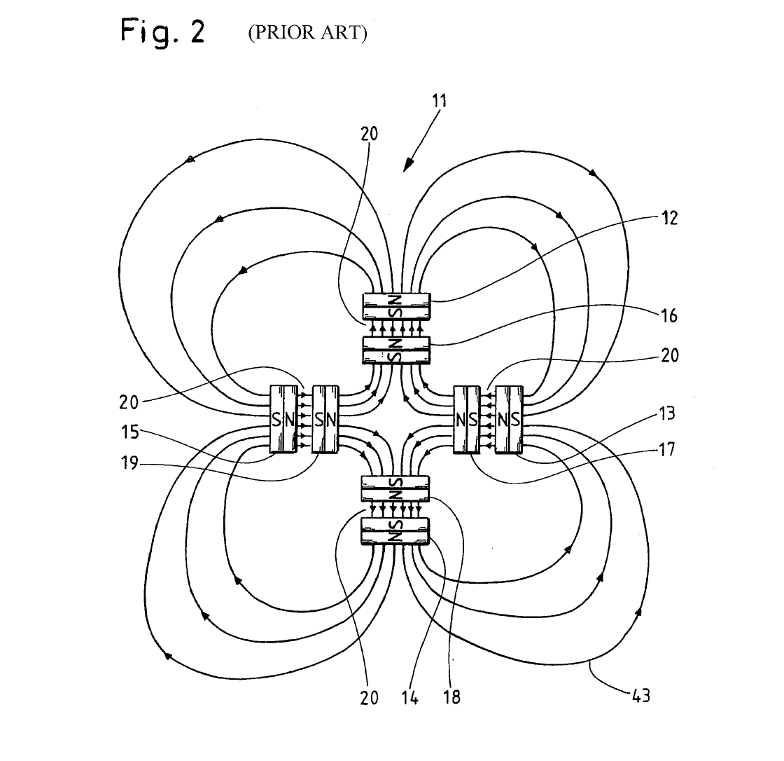

FIG. 2 shows in cross-section a magnetic arrangement of the known contact-free magnetic coupling according to FIG. 1, wherein further constructive details are omitted for clarity. The contact-free magnetic coupling 11 comprises a ring of outer bar magnets 12, 13, 14, 15 and an inner ring of bar magnets 16, 17, 18, 19. These are disposed pairwise, namely pairs of bar magnets 12 and 16, 13 and 17, 14 and 18, and 15 and 19. The pairs of magnets are each disposed with the same polarity. The pairs of magnets form a cross-shaped arrangement. On the whole, this results in a typical constellation of a quadrupole field.

Between the two bar magnets of each pair, there is a gap 20 in which the magnetic flux lines 43 are particularly concentrated, thus where a movement of the ring of the outer bar magnets 12 to 15 leads particularly effectively to the entrainment of the ring of bar magnets 16 to 19.

Correspondingly, more or fewer pairs of bar magnets can also be used. Preferably however, there is an even number of magnet pairs. The corresponding magnetic fields then with two pairs of bar magnets have the form of a dipole field, with four parts a quadrupole field, with six pairs a sextupole field and with eight pairs an octupole field, etc.

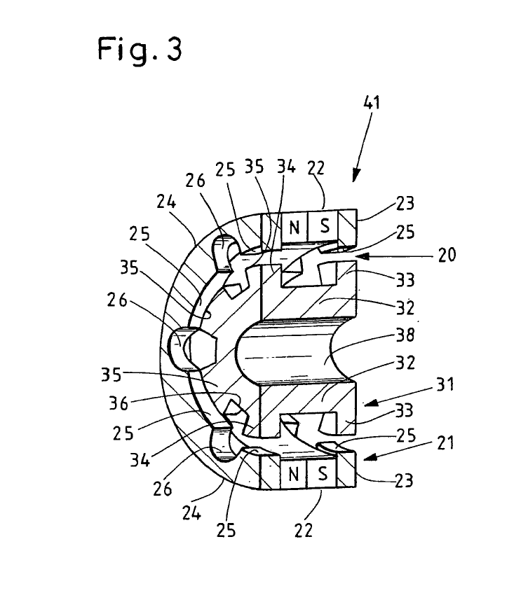

FIG. 3 shows a contact-free magnetic coupling 41 according to the invention in a schematic perspective drawing in an elevation. An outer coupling part 21 and an inner coupling part 31 are each configured substantially in the shape of annular bodies. Here, the outer coupling part 21 is constructed from an axially magnetized annular magnet 22, which is flanked or respectively bordered by two anchor plates 23, 24. The anchor plates 23, 24 are again annular disks. The outer periphery of the anchor plates 23, 24 corresponds to the outer periphery of the annular magnet 22, while the inner diameter of the anchor plates 23, 24 is smaller than the inner diameter of the annular magnet 22. As seen in FIG. 3, this results in a “U”-shaped cross section of the outer coupling part 21, wherein the “U” is open toward the interior, thus toward the center.

In the peripheral direction, the anchor plates 23, 24 also have recesses 26, each of which border a pole shoe segment 25. Due to this structure, the magnetic field lines, which are generated by the annular magnet 22, are preferably conducted through the inner surfaces of the pole shoe segments 25 of the anchor plates 23, 24 and exit from there. At these locations, the outer coupling part 21 has the “U”-shaped cross-section thereof.

An inner coupling part 31, which has a complementary shape to the coupling part 21, is disposed concentrically in the outer coupling part 21. Here, in the scope of the invention, a “complementary shape” is understood to be a functionally complementary shape. This means that the inner coupling part 31 has an annular body 32, which has substantially the same width as the outer coupling part 21. In addition, the annular body 32 in the example embodiment according to FIG. 3 has two flanking anchor plates 33, 34, which together with the annular body 32 results in this case in a U-shape open toward the exterior. The flanks, or respectively sides, of the “U”-shape of the inner coupling part 31 and the outer coupling part 21 point toward each other and lead therefore to a strong bundling of the magnetic field lines.

The inner coupling part 31 in FIG. 3 does not have an annular magnet of its own, rather is integral and produced from a ferromagnetic material. The anchor plates 33, 34 of the inner coupling part 31 again have recesses 36 in the peripheral direction that correspond to the recesses 26 in the anchor plates 23, 24 of the outer coupling part 21. Each of the recesses 36 of the anchor plates 33, 34 border in turn, pole shoe segments 35, which are across from pole shoe segments 25 of the anchor plates 23, 24. This results in a bundling of the magnetic field lines not only in the axial direction, but also in the peripheral direction. In this manner, a magnetic coupling is created between the outer coupling part 21 and the inner coupling part 31 in both the axial direction as well as in the rotational direction.

The inner coupling part 31 has a central opening 38, into which an inner tube 4 of an endoscope 1 is inserted. In the gap 20 between the inner coupling part 31 and the outer coupling part 21, there is for example, the continuation of the outer tube 3 in the handle 5 of the endoscope 1, similar to that in FIG. 1.

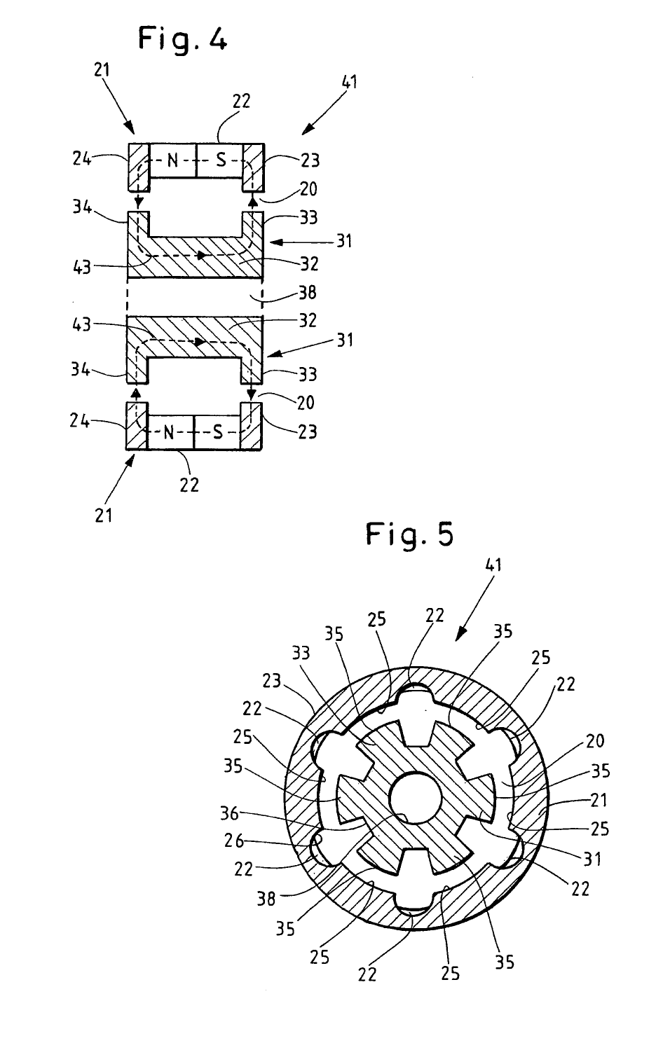

FIG. 4 shows in schematic cross section, a magnetic coupling 41 according to FIG. 3 together with exemplary magnetic field lines 43. Again it can be seen that the “U”-shape of the cross section of the inner coupling parts 31 and the outer coupling parts 21 together result in a ring closure which is interrupted only by the gap 20. The magnetic field lines that are generated by the annular magnets 22 are bundled and conducted by the ferromagnetic anchor plates 23, 24 and 33, 34 and the annular body 32, and are concentrated at the gap 20 between the anchor plates 23 and 33, or respectively 24 and 34.

In the scope of the invention it is not absolutely mandatory that the coupling part, which does not have any annular magnets, in FIG. 4 the inner coupling part 31, has projecting anchor plates 33, 34. It can also be merely a flat, cylindrical sleeve. The focusing of the magnetic field lines in the axial direction occurs then solely via the anchor plates 23, 24 of the outer coupling part 21. An axial transfer of force is guaranteed in this case too. However in such a case a structuring and force transfer in the peripheral direction is not possible, or only to a limited extent.

For the function of the contact-free magnetic coupling 41, it is not important whether the outer coupling part 21 or the inner coupling part 31 has the annular magnets 22. The situation can also be reversed such that the inner coupling part 31 comprises the annular magnets 22, while the outer coupling part 21, is in particular integral, produced from a ferromagnetic material, with or without anchor plates 23, 24. Likewise, a particularly strong coupling can be created in that annular magnets are disposed both in the inner coupling part 31 as well as in the outer coupling part 21.

FIG. 5 shows a schematic lateral representation of the contact-free magnetic coupling 41 according to the invention according to FIGS. 3 and 4. In the example embodiment shown here, the structuring of the anchor plates 23, 33 is shown in the form of the six pole shoes segments 25, 35 and the likewise six recesses 26, 36. With respect to the outer coupling part 21, the annular magnet 22 can also be seen through the recesses 26.

With reference to FIG. 4 and the view in FIG. 5 of the side with the anchor plates 33, 34, this is the visible side of the annular magnet 22, the side with the “south” polarity. The bundled field lines run through the gap 20 each preferably strengthened between the pole shoe segments 25 of the outer coupling part 21 and the opposite pole shoe segment 35 of the inner coupling part 31.

All named characteristics, including those taken from the drawings alone, and individual characteristics, which are disclosed in combination with other characteristics, are considered individually and in combination as essential to the invention. Embodiments according to the invention can be fulfilled through individual characteristics or a combination of several characteristics.

### REFERENCE LIST

1 endoscope
2 shaft
3 outer tube
4 inner tube
5 handle
6 distal end
7 entry window
8 prism unit
9, 9′ lens
10 CCD sensor
11 magnetic coupling
12-19 bar magnet
20 gap
21 outer coupling part
22 annular magnet
23, 24 anchor plate
25 pole shoe segment
26 recess
31 inner coupling part
32 annular body
33, 34 anchor plate
35 pole shoe segment
36 recess
38 central opening
41 magnetic coupling
43 magnetic field lines

What is claimed is:

1. A contact-free magnetic coupling for an endoscope, the contact-free magnetic coupling comprising:
an outer coupling part; and
an inner coupling part;
wherein the inner coupling part is disposed within the outer coupling part such that a gap remains between the outer and inner coupling parts;
the outer coupling part and the inner coupling part each comprise an annular body; and
wherein the annular body of the outer coupling part is disposed between first side anchor plates, which together form a substantially “U”-shaped cross section that is open toward an interior; and/or
the annular body of the inner coupling part is disposed between second side anchor plates, which together form a substantially “U”-shaped cross section that is open toward an exterior;
wherein the annular body of the outer coupling part and/or the inner coupling part comprises an axially magnetized annular magnet.

2. The contact-free magnetic coupling according to claim 1, wherein one of the inner and outer coupling parts which does not comprise an annular magnet is composed at least to some extent from a ferromagnetic material.

3. The contact-free magnetic coupling according to claim 2, wherein the one of the inner and outer coupling parts which does not comprise an annular magnet is formed of one-piece.

4. The contact-free magnetic coupling according to claim 1, wherein each of the inner and outer coupling parts have axially magnetized annular magnets which are axially poled oppositely to each other.

5. The contact-free magnetic coupling according to claim 1, wherein the first and/or second anchor plates are composed at least to some extent from a ferromagnetic material.

6. The contact-free magnetic coupling according to claims 1, where the first and/or second anchor plates of the outer and inner coupling parts, respectively, at surfaces thereof bordering the gap between the outer and inner coupling parts have a structure in the peripheral direction with pole shoe segments corresponding to each other.

7. The contact-free magnetic coupling according to claim 6, wherein the first and second anchor plates of each of the outer and inner coupling parts, respectively, have the same shape.

8. The contact-free magnetic coupling according to claim 6, wherein the first and second anchor plates of each of the outer and inner coupling parts, respectively, are disposed in the same angular relationship to each other.

9. The contact-free magnetic coupling according to claim 6, wherein the first and second anchor plates of each of the outer and inner coupling parts have different shapes.

10. The contact-free magnetic coupling according to claim 9, wherein the first and second anchor plates of each of the outer and inner coupling parts have different numbers of pole shoe segments

11. The contact-free magnetic coupling according to claim 9, wherein the first and second anchor plates of each of the outer and inner coupling parts are disposed in a different angular relationship to each other.

12. An endoscope having a contact-free magnetic coupling according to claim 1.

13. The endoscope according to claim 12, wherein the endoscope is a video endoscope.

14. The endoscope according to claim 12, wherein the endoscope has a viewing direction that can be diverted.

15. The endoscope according to claim 12, wherein the endoscope has a viewing direction that can be changed.

16. The endoscope according to claim 12, wherein the endoscope has a changeable sideways viewing direction.
